(19) United States
(12) Patent Application Publication
(10) Pub. No.: US 2024/0312575 A1
(43) Pub. Date: Sep. 19, 2024

(54) SYSTEM AND METHOD FOR AUTOMATICALLY DETERMINING SERIOUS ADVERSE EVENTS

(71) Applicant: Medidata Solutions, Inc., New York, NY (US)

(72) Inventors: Jingshu Liu, New York, NY (US); Robert Buka, New York, NY (US); Patricia Allen, Boston, MA (US); Hrishikesh Karvir, Langhorne, PA (US); Michael Elashoff, San Mateo, CA (US)

(21) Appl. No.: 18/670,625

(22) Filed: May 21, 2024

Related U.S. Application Data
(63) Continuation of application No. 17/108,901, filed on Dec. 1, 2020.

(51) Int. Cl. G16H 10/20; G06N 3/049; G06N 20/00; G16H 50/20

(52) U.S. Cl. CPC G16H 10/20; G06N 3/049; G06N 20/00; G16H 50/20

(57) ABSTRACT

Detecting serious adverse events from Electronic Data Capture (EDC) data for clinical trials. A method includes extracting clinical data from EDC data of a plurality of clinical trials, the clinical data comprising form names and field names. The clinical data is standardized across the plurality of clinical trials to generate subject-level data and adverse event data. The adverse event data is used to produce standardized adverse events. A serious adverse event model is trained by inputting the standardized adverse events, trial-level features, and the subject-level data. The trained serious adverse event model is used to determine a probability that a standardized adverse event is a serious adverse event.

### CROSS-REFERENCE TO RELATED APPLICATION(S)

This application is a continuation of U.S. patent application Ser. No. 17/108,901, filed on Dec. 1, 2020, which is incorporated herein by reference.

### BACKGROUND

Clinical trials (sometimes called “clinical studies”) are often used to assess the safety and efficacy of a drug or a medical device. In some trials, hundreds or thousands of test sites enroll thousands or tens of thousands of subjects or patients.

One metric that may be monitored during a clinical trial is the occurrence of adverse events, sometimes abbreviated “AE.” An AE typically includes any event that is experienced by a clinical trial subject during his/her participation in the trial that may have a negative impact on health or well-being, such as headache, stomachache, dry mouth, high blood pressure, fast heart rate, migraines, seizures, stroke, heart attack, etc.

A specific type of AE is a serious adverse event or “SAE.” An adverse event is considered serious if, according to the clinical trial investigator, the outcome of the event is any of the following: death, a life-threatening event, inpatient hospitalization (initial or prolonged), disability, significant incapacity to conduct normal life functions, congenital anomaly or birth defect, or other important medical events that may lead to one of the aforementioned outcomes. It is critical to be able to quickly detect and/or determine SAEs that occur during a clinical trial to prevent other clinical trial subjects from suffering from the same SAE or at least to understand when such an SAE may occur.

### BRIEF DESCRIPTION OF THE DRAWINGS

FIG. 1 is a flowchart showing one process by which adverse events are currently reviewed and determined;

FIG. 2 shows the top ten most frequently occurring adverse event terms along with their frequency and the percentage of time they are considered serious;

FIG. 3 shows the top ten adverse event terms along with their frequency and the percentage of time they are considered serious;

FIG. 4A is block diagram of a system for developing and using a model to automatically detect probable serious adverse events, according to an embodiment of the present invention;

FIG. 4B illustrates a process used by the model developer of FIG. 4A to develop a model, according to an embodiment of the present invention;

FIG. 5A is a diagram showing the embedding of high-dimensional input variables as a continuous vector to be used by the neural network, according to an embodiment of the present invention;

FIG. 5B is a diagram showing one-hot encoding of low-dimensional features and normalization of other numerical features, according to an embodiment of the present invention;

FIG. 6A is a diagram of a feed-forward deep learning model, according to an embodiment of the present invention;

FIGS. 6B and 6C are diagrams of bi-directional, long-short term memory deep learning models, according to embodiments of the present invention;

FIG. 7A is a diagram showing precision-recall curves for various tested models against the benchmark model, according to embodiments of the present invention;

FIG. 7B is a diagram showing receiver operating characteristic curves for various tested models, according to embodiments of the present invention;

FIG. 7C is a diagram showing SAE coverage by review amount for the benchmark model and the bi-directional LSTM model, according to embodiments of the present invention;

FIG. 8 is a diagram showing how the results of embodiments of the present invention are presented in a user interface;

FIGS. 9A and 9B show how embodiments of the present invention may be used to prioritize adverse event review; and

FIG. 10 is a flowchart showing how the serious adverse event model of the present invention can be integrated into the adverse event review process.

Where considered appropriate, reference numerals may be repeated among the drawings to indicate corresponding or analogous elements. Moreover, some of the blocks depicted in the drawings may be combined into a single function.

### DETAILED DESCRIPTION

In the following detailed description, numerous specific details are set forth in order to provide a thorough understanding of embodiments of the invention. However, it will be understood by those of ordinary skill in the art that the embodiments of the present invention may be practiced without these specific details. In other instances, well-known methods, procedures, components, and circuits have not been described in detail so as not to obscure the present invention.

A sponsor (i.e., the drug company that makes the drug being tested) is responsible for ongoing safety evaluation of investigational products. Sponsors are thus required by the FDA and other regulatory bodies to submit reports of Serious and Unexpected Suspected Adverse Reactions (SUSARs) to all investigators, ethics committees, and competent authorities. The information included in such safety reports has to be consistent, accurate and complete. It has been reported that the accuracy and completeness of SAE case reports are poor, which can delay identifying safety signals. See, e.g., S. Crépin et al., Pharmacoepidemiol. Drug Safety 2016; 25: 719-24.

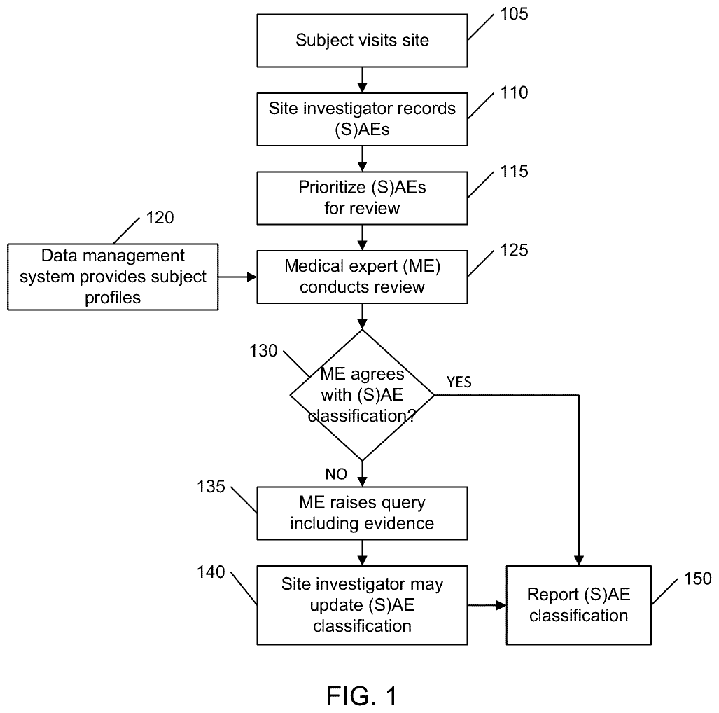

Reference is now made to FIG. 1, which is a flowchart showing one process by which adverse events are currently reviewed and determined. In operation 105, a subject visits a clinical trial site, and, in operation 110, a site investigator records whether that subject has had an adverse event, serious or not. In operation 115, the Sponsor prioritizes the adverse events for review based on perceived seriousness. One indication of seriousness is the severity grade of the event on a five-level adverse event scale developed by the National Cancer Institute. On this scale, Grade 1 is a mild AE (from the inventors' data, less than 1% are serious); Grade 2 is a moderate AE (approximately 6.5% are serious); Grade 3 is a severe AE (approximately 41% are serious); Grade 4 is a life-threatening or disabling AE (approximately 47% are serious); and Grade 5 is a death related to the AE (over 99% are serious). See NCI Common Terminology Criteria for Adverse Events v3.0, August 2006 (“CTCAE”). Another indication of seriousness is the e.g., existence of the event on the Important Medical Events (IME) list developed in 2007 by the EudraVigilance Expert Working Group of the European Medicines Agency (EMA). Another indication of seriousness is hospitalization records.

In operation 125, a medical expert conducts a review of the reported seriousness of adverse events based at least in part on a data management system operation 120 that provides subject profiles. An example of such a system is JReview®, provided by Integrated Clinical Systems, Inc. This medical review of trial data, both cumulative data and individual subject data, tries to identify potential issues that could affect either the safety of trial subjects or the progress of the trial. The medical review includes an ongoing, real-time review per subject, as well as a periodic, comprehensive review across subjects at specific time points (e.g., at the Data Monitoring Committee (DMC) meeting, which occurs prior to a final, blind review meeting) for plausibility and consistency from a medical perspective as planned in the Medical Review Plan (MRP). The medical review supports the medical quality of the clinical data (e.g., efficacy and safety data). The medical review is based on data from the clinical database (e.g., data sets/tables/listings) and on pharmacovigilance data (e.g., CIOMS (Council for International Organizations of Medical Sciences) and blinded SUSAR reports) in format and content as specified in the MRP. Operation 130 asks whether the medical expert agrees with the site investigator's determination of whether an adverse event is serious or not. If so, then in operation 150 the adverse event is reported with the site investigator's determination of seriousness. If the medical expert does not agree with the determination, the expert in operation 135 raises a query to the site investigator including evidence to support the expert's determination. In operation 140, the site investigator then may or may not update the determination of seriousness based on the expert's query. (The decision is ultimately up to the site investigator. The sponsor's medical expert can query and re-query to urge the investigator to reclassify, but the medical expert cannot override the site investigator.) The resulting event is then reported in operation 150.

Another method used to determine the seriousness of adverse events is the EMA's IME list, as described above. The list identifies preferred terms (PTs) from the Medical Dictionary for Regulatory Activities (MedDRA®), developed by the International Conference on Harmonisation of Technical Requirements for Registration of Pharmaceuticals for Human Use (ICH), that are medically important regardless of the presence of other regulatory seriousness criteria. The list's purpose is to facilitate the classification of suspected serious adverse reactions, to aggregate data analysis, and to perform case assessment for pharmacovigilance activities. Some pharmaceutical companies use this list to identify potential serious adverse events in clinical studies. It is updated with each MedDRA version (twice a year) and is based on a Medicines and Healthcare products Regulatory Agency (MHRA) list. It is intended for guidance purposes only: there is no mandatory requirement for regulatory reporting and there is an option to use the list for other purposes.

There are challenges in detecting and/or determining SAEs using these current methods. First, the current methods are highly manual and subjective processes. Second, there are different standards within and among organizations in determining what is an SAE. Third, SAE detection and/or determination suffers from systemic and human error, time-consuming and costly review, and frequent lack of quantitative evidence.

The inventors have developed a system and method to address these challenges by automatically and reliably determining SAEs. The system and method use a data-driven approach that provides quantitative evidence to investigators and sponsors during the medical review process. The system and method improve both the accuracy and efficiency of SAE reporting. Using the data-gathering platform developed by Medidata Solutions, Inc., the assignee of the present invention, the inventors identified and used patterns from more than one million adverse event records, including nearly 70,000 serious adverse events, from over 1,800 clinical trials completed since 2007. The benefits of the present invention include improving the probability of detecting and/or determining SAEs without reducing the probability of correctly classifying non-serious AEs, prioritizing (S)AEs for medical review with greater precision, and providing quantitative and objective evidence of the seriousness of adverse events.

In addition to distinguishing among types of adverse events to determine whether one is serious, the invention also addresses determining whether an adverse event, which is sometimes serious and sometimes not, qualifies as an SAE. Patterns begin to emerge once all the data are pooled together and the terms used to describe the adverse events are standardized. From data reviewed by the inventors, over 3,850 total terms have at least one serious observation, but the same event may or may not always be categorized as serious or non-serious. The differences in categorization may be due to other contextual features, e.g., the severity of the event, the age of the subject, the indication of the drug under investigation, etc. For example, a “headache” may be considered serious in a trial for a brain cancer treatment but perhaps not considered serious in a trial for an allergy treatment.

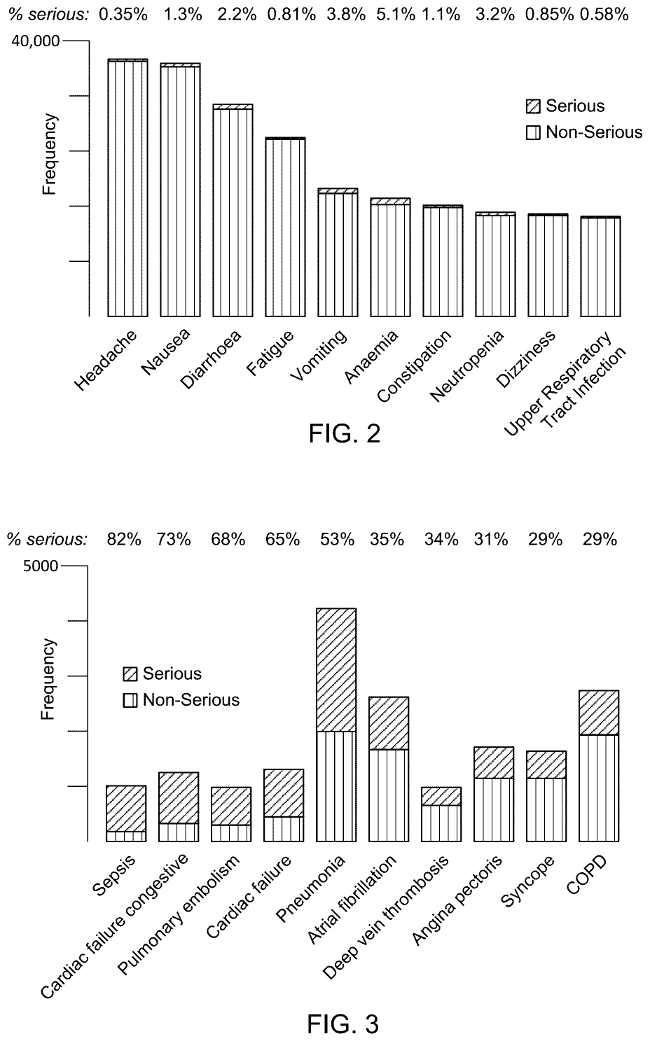

FIG. 2 shows the top ten most frequently occurring adverse event terms along with their frequency and the percentage of time they are considered serious. Even a headache can sometimes be considered an SAE. FIG. 3 shows the top ten serious adverse event terms captured in Medidata Solutions, Inc.'s data-gathering platform along with their frequency and the percentage of time they are considered serious. These top ten terms encompass ˜12.5% of all serious observations.

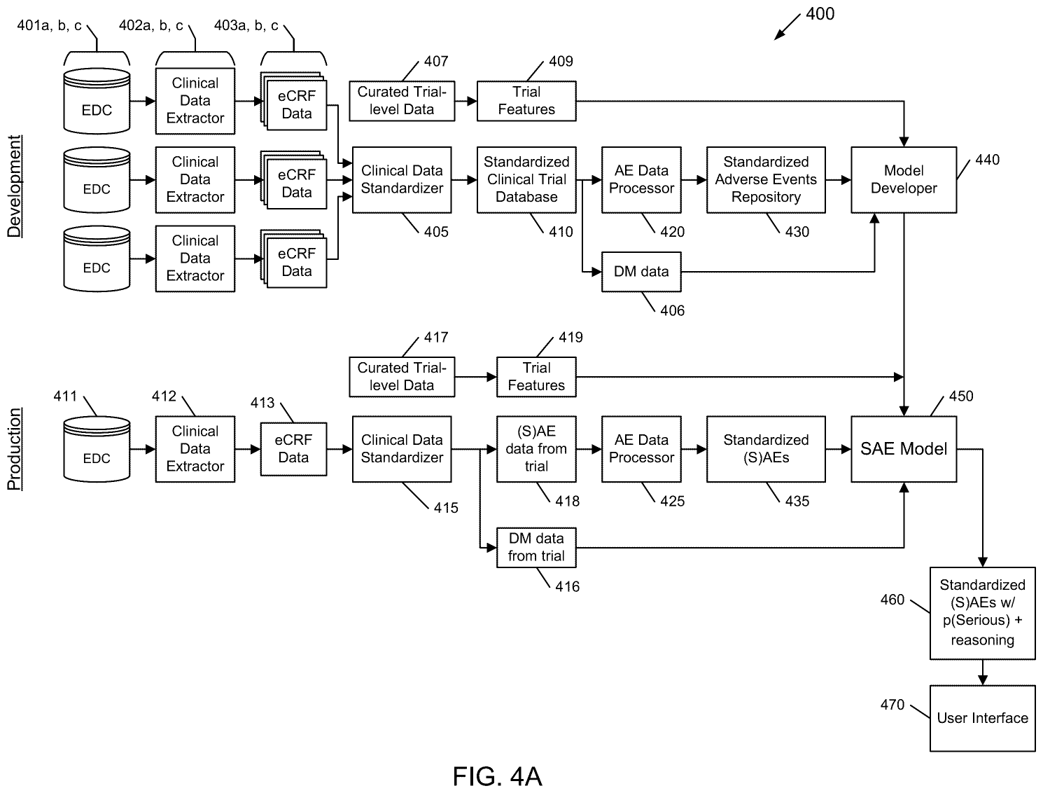

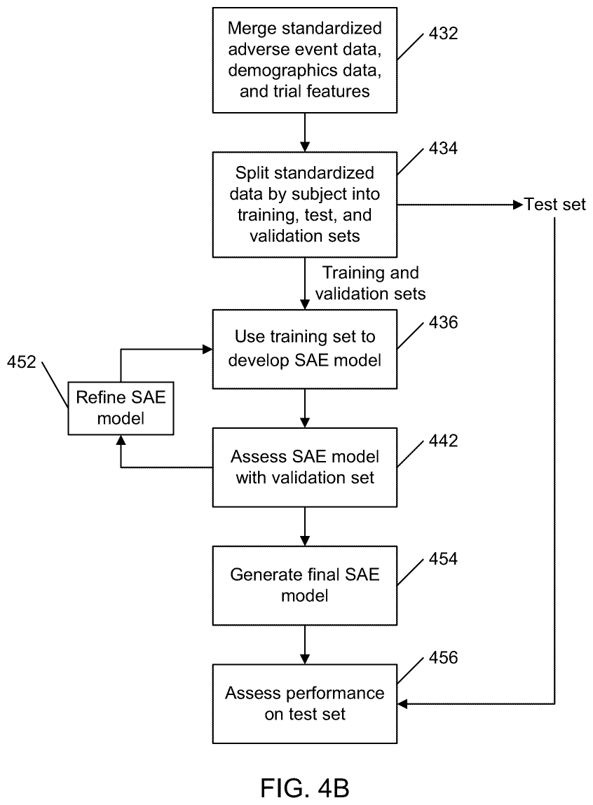

Reference is now made to FIGS. 4A and 4B, which illustrate several aspects of the present invention. FIG. 4A is a block diagram of a system 400 for developing a model to automatically detect probable serious adverse events, according to an embodiment of the present invention. FIG. 4A includes production and development workflows. The development workflow illustrates the development of one or more SAE models to be used to determine the seriousness of an adverse event. The production workflow illustrates how the SAE models are used with real clinical trial data to assess seriousness of adverse events.

Part of the utility of this invention comes from standardized clinical trial database 410, which is very comprehensive and includes data from thousands of clinical trials, hundreds of sites, multiple therapeutic areas, and multiple sponsors. As the size of the database increases, the benefits of the invention are more easily realized.

The development workflow first includes clinical data standardizer 405, to which are input data from multiple clinical trials are input and which standardizes the data and form and field names across the clinical trials. These data come from EDC 401a, b, c, which indicates electronic data capture from multiple clinical trials (of which three are indicated in FIG. 4A). An example of EDC 401 is the Medidata Rave® EDC system. Clinical data extractors 402a, b, c then extract the relevant clinical data from EDC 401a, b, c and generate the raw eCRF (electronic case report form) data 403a, b, c. The eCRF data are then input to clinical data standardizer 405, which standardizes the data and form and field names across the clinical trials and determines from which forms and fields data may be extracted by Adverse Event (AE) data processor 420. For example, the output of clinical data standardizer 405 provides the following information for study XYZ:
form name: “AEx”=Adverse Event form
field name: “AETERM”=Adverse Event field and for study ABC:
form name: “AEy”=Adverse Event form
field name: “EVENT”=Adverse Event field
   With these outputs from clinical data standardizer 405, the data associated with AE forms and fields (or form and field names) can be extracted and then pooled together across trials in the standardized adverse events repository. Clinical data standardizer 405 may use, for example, a form and field classifier that pools together the corresponding data for analysis. The outputs from clinical data standardizer 405 may then be stored in standardized clinical trial database 410 or may be input directly to AE data processor 420.

AE data processor 420 processes the standardized data from clinical data standardizer 405 and/or standardized clinical trial database 410, including the standardized form and field names, to generate standardized adverse event terms, and stores/pools them together across trials in standardized adverse events repository 430, along with other adverse event-level data described below. Storing adverse events using standard terms improves the input to model developer 440. One type of such processor is disclosed in U.S. patent application Ser. No. 15/443,828, filed Feb. 27, 2017, assigned to Medidata Solutions, Inc., the assignee of the present invention, and incorporated herein by reference in its entirety. That application discloses an apparatus and method for automatically mapping verbatim narratives to terms in a terminology dictionary. In contrast to some types of clinical trial data, such as blood pressure and heart rate, which may be recorded as numbers, an adverse event that occurs during a clinical trial is generally recorded as a text or verbatim narrative. The format of such narratives may differ from one recorder (e.g., subject, doctor, nurse, technician, etc.) to another and may even differ by the same recorder at a different time. Such differences may be as simple as spelling differences, which may be caused by typographical errors or that some words are spelled differently in different geographic areas. One example is “diarrhea,” which may be misspelled (e.g., diarrea) or may be spelled differently in different countries (e.g., in the United Kingdom it is spelled “diarrhoea”). Other times, the same condition is described by its symptoms rather than a specific name. So “diarrhea” may be described as “loose stools,” “Soft bowels,” “soft stools,” “Loose bowel movements,” etc., and each of these words may be capitalized, may appear in singular or plural, or may be misspelled. The processing maps each narrative to a term or terms in a terminology dictionary, such as MedDRA.

Standardized adverse event data are input to model developer 440, along with other attributes such as demographic information or data 406 and trial features 409. Adverse event-level data or information (also called “features”) include the adverse event MedDRA preferred term (PT), the severity grade (on the NCI's five-level scale), the duration of the event (e.g., if the subject recovered in one day or less, which may indicate lesser seriousness), whether the adverse event is on the IME list, and the adverse event's relationship to the trial treatment or AEREL. (This is a standard field in the SDTM (Study Data Tabulation Model), which is an industry standard data model for clinical trial data. AEREL is the relationship of the adverse event to the treatment that was under investigation in that particular trial, i.e., an indication of whether the trial treatment had a causal effect on the adverse event, as reported by the clinician/investigator.)

Subject-level demographic data or information (also called “features”) 406 include age, gender, race, concurrent events (including severity and seriousness) experienced by the subject on the same day, and previous events (including severity and seriousness) experienced by the subject. Trial-level features 409, which come from a table containing curated trial-level data 407, include indication, phase, sponsor, therapeutic area, and the primary purpose of the trial reported to the NIH on clinicaltrials.gov. Other features may include hospitalization, lab values, medical history, and concomitant medications. These features are not exhaustive; others may be used in addition or instead.

Model developer 440 evaluates several models to develop a multivariate, probabilistic SAE model 450 for use in determining whether an adverse event is serious. The models considered herein include a benchmark model (i.e., rules-based algorithm using IME list+AE Severity), logistic regression, extreme gradient boosting (“XGBoost”), and neural network models such as feed-forward and bi-directional, long(-term) short-term memory (Bi-LSTM), both of which will be discussed in detail below.

Reference is now made to FIG. 4B, which depicts a more detailed process used by model developer 440 to develop an SAE model, according to an embodiment of the present invention. Operation 432 merges standardized adverse event data (which includes standardized adverse event terms and other adverse event data such as severity and relationship to treatment) from standardized adverse events repository 430, demographic data 406, and trial features 409 into merged, standardized data. Operation 434 splits the merged, standardized data into a training set and a test set, randomly by subject (i.e., the same subject does not appear in different sets), or into a training set, a test set, and a validation set. In one embodiment, 80% of the events are used in the training set and 20% are used in the test set (or 20% are used in the test and validation sets), but other ratios may be used, such as 75%:25% (or 75%:15%:10%) or 70%:30% (or 70%:15%:15%), and the validation and training sets do not have to be the same size. Typically, however, the training set includes most of the records. Operation 436 uses the training set to develop an initial SAE model. This operation assesses a variety of model parameters including model architecture, e.g., for Bi-LSTM, the number of hidden layers and the number of nodes within each hidden layer, and feature engineering. Here, the parameters (or weights or coefficients) of the model are updated iteratively, so that the output of the model becomes closer and closer to the observations in the training set. Closeness may be measured by statistical metrics such as mean-squared error or classification accuracy.

The details of the models considered will now be described. The first is a benchmark or baseline model. This model follows the rule that an adverse event is classified as an SAE (score=1) if the AE is on the IME list or the severity is Grade 3 (severe) or higher on the CTCAE scale, otherwise it is classified as a non-SAE (score=0). This baseline reflects industry practice to some extent, as some sponsors use the severity grade and/or the IME list to provide directional guidance in reviewing events. Although this model is called a “benchmark,” this approach does not generate a probability score as other approaches. It treats events on the IME list or with different severity scores (3 and above) as having the same seriousness likelihood. This is often not a realistic assumption. In addition, this model does not consider any other factors such as the target disease or indication of the trial. These drawbacks provide motivation to test a range of probabilistic modeling approaches, as described herein.

The next model is a machine learning model that uses logistic regression with manually engineered features. That is, the interactions between features are manually specified (i.e., input to the formula of the model manually) and tested. So, several of these models were tested, each with a different combination of features. The features used in all of the models included the five-level severity score and the MedDRA preferred term. Later models added one or more of the indication of the trial drug, outcome of the adverse event, the subject's age, the average seriousness of previous occurrences, and the sponsor name. Another feature that may be used (in this model and in the XGBoost model described below) is the percentage of time that an adverse event of the same type and severity grade is labeled as serious.

The next model, also a machine learning model, extreme gradient boosting (“XGBoost,” see Tianqi Chen and Carlos Guestrin, “XGboost: A scalable tree boosting system,” Proceedings of the 22nd ACM SIGKDD International Conference on Knowledge Discovery and Data Mining, ACM, 2016), offers benefits over logistic regression, because it can automatically model the interactions between features. This model uses an ensemble classification and regression tree (CART) method, using boosting/additive training (with a gradient boosting machine, GBM) as well as random subsampling of samples and features (random forest algorithm). The XGBoost model includes regularization on the complexity of trees, but requires feature engineering, e.g., converting variables of high-cardinality to numerical values (or one-hot encoding), to model the relationships between the features. Like logistic regression, several of these models were tested, using the same combinations of features as logistic regression—five-level severity score, MedDRA preferred term, indication of the trial drug, outcome of the adverse event, the subject's age, the average seriousness of previous occurrences, and the sponsor name.

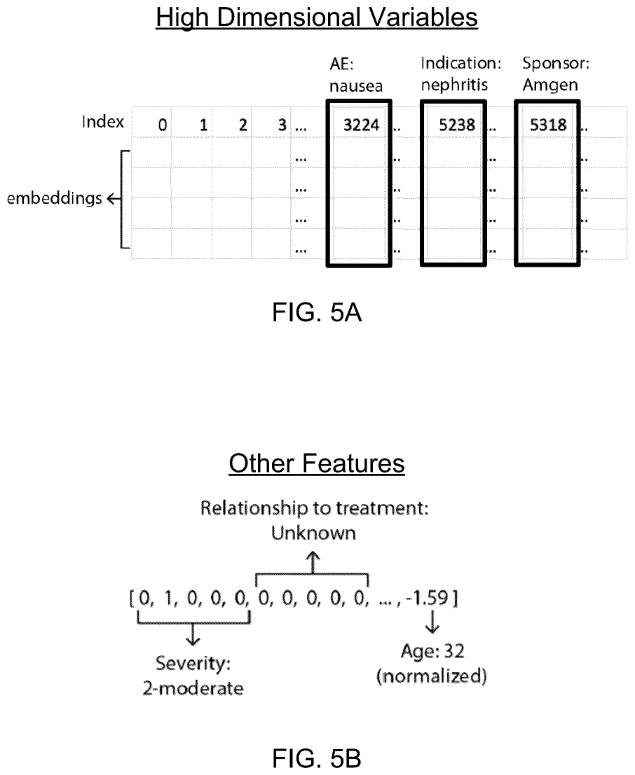

The next models are neural networks (deep learning models), such as feed-forward, Bi-LSTM, and convolutional (whose performance was similar to feed-forward). The deep learning models learn the complex interactions between variables. They have a flexible architecture to incorporate different sources of information, whereas logistic regression models typically manually define and select the interaction terms. The deep learning models learn vectorized representations of high-dimensional variables, whereas XGBoost models typically manually transform high-dimensional variables to numerical values. The input structure for the deep learning models represents each event or high-dimensional variable (or group of same-day events) as embedded numerical vectors, as seen in FIGS. 5A and 5B. FIG. 5A shows the embedding of high-dimensional input variables, e.g., AE preferred term (PT), Indication, and Sponsor, as a continuous vector to be used by the neural network. FIG. 5B shows the one-hot encoding of features that are low-dimensional by definition, e.g., AE Severity, AE Relationship to Treatment, as well as the normalization of numerical features such as Age. One-hot vectors are binary vectors that include all zero values except at the index of the categorical value marked with a 1, for example, severity grade 2=[0,1,0,0,0].

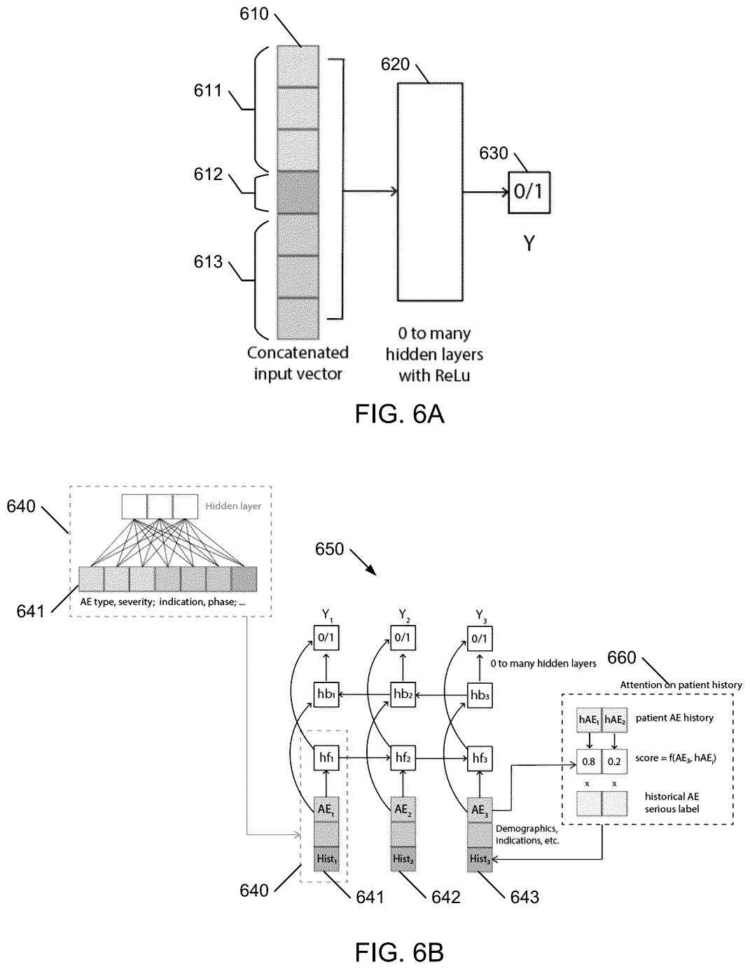

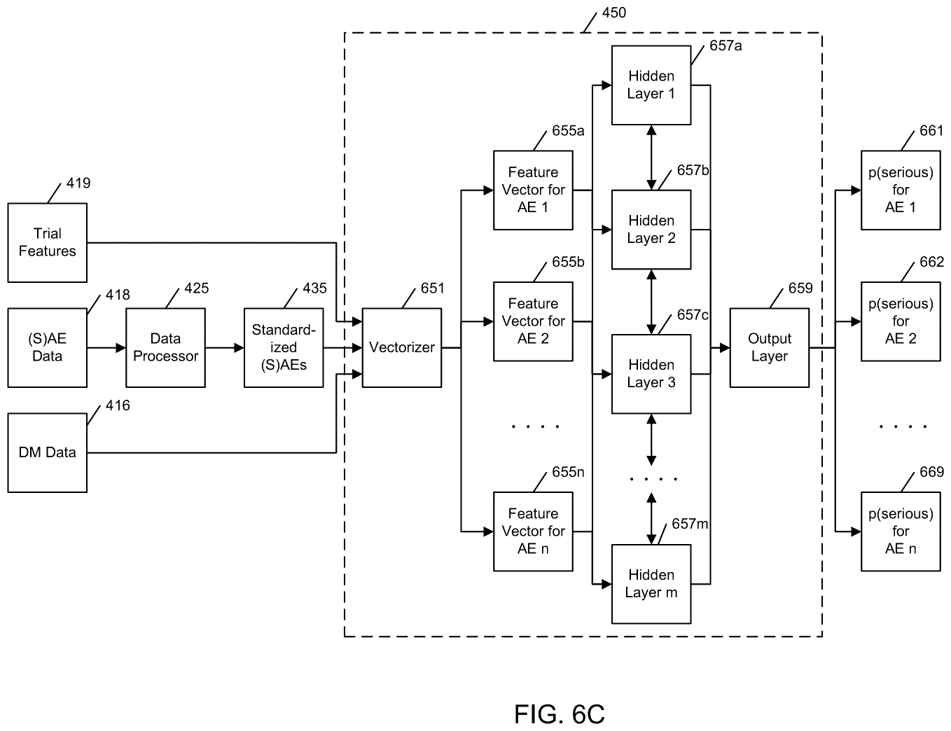

One deep learning model is a feed-forward model, illustrated in FIG. 6A. In such a model, a concatenated input vector 610 may be made up of subvectors 611, 612, 613. A concatenated vector includes embedded/encoded features. Subvector 611 may include adverse event-level features as earlier listed (e.g., severity, preferred term); subvector 612 may include trial-level features as earlier listed (e.g., indication, phase); and subvector 613 may include subject-level features as earlier listed (e.g., age, gender, previous AEs). The subvectors may also include a URL that represents a collection of studies by a particular sponsor or sponsor plus Contract Research Organization/partner pair. These subvectors are interchangeable, i.e., subvector 611 is not specific to AE features, subvector 612 to trial-level features, and subvector 613 to subject-level features. FIGS. 5A and 5B can be viewed as components of subvectors 611 and 613. Subvector 611 can be constructed by concatenating several vectors from FIG. 5A (one for AE, Indication, and Sponsor respectively), and subvector 613 can be a concatenation of several vectors of FIG. 5B (one for each categorical variable).

Block 620 indicates the number of hidden layers and that each hidden layer has a ReLU (rectified linear unit) activation function. The network makes a decision Y, in block 630, of either a 0 or a 1. One example of the architecture of a feed-forward model is to use 50 embedding dimensions, together with other numerical features that adds up to an input vector dimension of 183. The input vector is then connected to a hidden layer with 512 nodes, the output is then connected to a hidden layer with 256 nodes, which is then connected to an output layer with 1 node.

Another deep learning model is a Bi-LSTM model. An LSTM (long(-term) short-term memory) model is a type of recurrent neural network (RNN) used in sequence modeling problems such as language processing. The model takes inputs sequentially and updates the model's internal representation at each step, based on both the current input and the previous representation. See Sepp Hochreiter and Jürgen Schmidhuber, “Long short-term memory,” Neural Computation 9(8): 1735-1780 (1997). A Bi-LSTM model is an extension of the standard LSTM model because it is bi-directional. See Alex Graves, Abdel-rahman Mohamed, and Geoffrey Hinton, “Speech recognition with deep recurrent neural networks,” 2013 IEEE International Conference on Acoustics, speech and signal processing (ICASSP) (IEEE 2013). Such a model combines two LSTMs where one is running forward in time and the other is running backward. The context window around each input thus consists of both information prior to and after the current input. A bi-directional LSTM is used to model complicated relationships between concurrent events. In the present invention, the adverse events of each subject are sorted by starting date and separated into events occurring on the current (i.e. same) day (to be evaluated for seriousness likelihood) and events that occurred previously (to be used as additional model inputs).

One embodiment of a Bi-LSTM model is illustrated in FIG. 6B. Block 640 shows hidden layers connected to concatenated input vector 641 as described with respect to input vector 610 and hidden layers 620 in FIG. 6A. This block learns the interactions between the features of each adverse event because the model architecture (hidden layers and their connections) allows one input to interact with another as data propagates through the hidden layers. “Learn” here means the automatic updating of the parameters used in those hidden layers in the training process. Once the size and number of layers are defined upfront, these parameters are not manually adjusted. Adverse events 1, 2, 3, etc. (AE₁, AE₂, AE₃, etc.), which occurred on the same day, and their associated features are used as sequential inputs to block 650. Hidden representation in the model is updated based on both the current input and the previous hidden states, either starting from left to right (hf₁, hf₂, etc.) or starting from right to left (hb₁, hb₂, etc.). Then an SAE likelihood score is generated based on both of the hidden representations (i.e., forward and backward) for each event.

More specifically, each event input consists of dense vectors 641, 642, 643 (embeddings) (where a dense vector has continuous values rather than just 0 or 1) representing the event preferred term (PT), trial indication and sponsor, and one-hot encoded vectors representing the five-level severity grade, the event's relationship to treatment, subject race, gender, trial phase, and primary purpose, and values of the remaining numerical features. The embeddings of the event, indication, and sponsors can either be learned together with other parameters in the model, or pre-trained. The input also has a value based on a subject's previous events (Hist₁, Hist₂, etc.) (i.e., attention on subject history). The value is computed in block 660 by a weighted sum of seriousness labels (SAE=1, non-SAE=0) of all previous events (hAE₁, hAE₂, etc.) that happened to the same subject. The weight is computed by the dot-product of the current event (concatenated event embedding and severity grade) and each previous event, and normalized across previous events so the weights sum to one. This block thus learns how to use subject history without manual feature engineering.

One example of the architecture of a Bi-LSTM model is to use 50 embedding dimensions (as in the feed-forward model) and 181 input vector dimensions. There can be multiple LSTM gated recurrent unit (GRU) hidden layers per direction, while the embodiment of this application uses 1 hidden layer per direction with 256 nodes each. The concatenated forward and backward hidden layers, with a dimension of 512, are then connected to the output layer of dimension 1. Compared to the feed-forward model, the Bi-LSTM model considers all same-day adverse events together (where adverse events occurring within three days apart are treated as same-day events) and learns how to use AE history, instead of feature engineering.

FIG. 6C is a detailed block diagram of the Bi-LSTM model, according to an embodiment of the present invention. Via vectorizer 651, SAE model 450 (see FIG. 4A) takes as inputs trial features 419, DM data 416, and standardized (S)AEs 435, which result from SAE data 418 being processed by data processor 425. Vectorizer 651 embeds the features of each adverse event record into a single feature vector 655 (as described with respect to FIGS. 5A and 5B) to be used by the neural network (as described with respect to FIG. 6B). The neural network is composed of a number (e.g., m) hidden layers 657a, 657b, 657c, . . . 657m and output layer 659 as described in the previous paragraph. As the feature vector 655a, 655b, . . . 655n for each event passes through the hidden layers, the hidden representation is updated based on both the current input and the previous hidden states, either starting from left to right (hf1, hf2,etc.) or starting from right to left (hb1, hb2, etc.). Then an SAE likelihood score 661, 662, . . . 669, i.e., the probability of seriousness for each adverse event, is generated based on the forward and backward hidden representations for each adverse event.

The blocks shown in FIGS. 4A and 6C are examples of modules that may comprise the various systems described and do not limit the blocks or modules that may be part of or connected to or associated with these modules. For example, FIG. 4A shows trial features 409, demographic data 406 and standardized adverse event data from standardized adverse events repository 430 being input to model developer 440. Alternatively, these three types of data may be merged before being input to model developer 440. Moreover, while AE data processors 420, 425 are shown as blocks separate from other blocks, they may be implemented in a general data processor that performs other functions. The blocks in these Figures may be implemented in software or hardware or a combination of the two, and may include memory for storing software instructions.

Operation 442 assesses the SAE model. The model architecture that is ultimately selected, as well as hyper-parameters for the model, are assessed based on the validation set performance. Model hyper-parameters may include the number of hidden layers or the number of nodes within each hidden layer. For each hyper-parameter set, a model is trained on the training set data (in operation 436), and the resulting model is used to generate an SAE probability score for each AE in the validation set. These predicted probability scores are compared to actual “serious” labels in the data for each parameter set described in operation 436. (The “serious” labels are reported during the trial.) This operation in effect selects the optimal hyper-parameter set.

The performance of the model is summarized for each set using two area under curve (AUC) metrics—the area under the receiver operating characteristic (ROC) curve (or AUROC) and the area under the precision-recall (PR) curve. These areas are used to compare performance and select the best model with the highest AUCs.

Both of these AUC metrics take into account a combination of true positives, false positives, false negatives, and true negatives. With respect to precision and recall, precision is the number of true positives divided by the sum of the true positives and false positives:

$$Precision = \frac{T P}{T P + F P}$$

Precision measures the percentage of predicted SAEs that are actually SAEs (i.e., that are actually reported serious in the trial). Precision describes how good a model is at predicting the positive class, i.e., precision describes the percentage of the results that are relevant. (“Positive” class=class 1=SAE; “negative” class=class 0=nSAE or non-serious AE.) Recall is the number of true positives divided by the sum of the true positives and false negatives:

$$Recall = \frac{T P}{T P + F N}$$

Recall measures the percentage of actual SAEs that were predicted to be SAEs. Recall describes the percentage of total relevant results correctly classified by the algorithm. Reviewing both precision and recall is useful in cases in which there is an imbalance in the observations between two classes, in this case a non-serious AE (nSAE) class (class 0) and an SAE class (class 1). Specifically, here there are many examples of nSAE (class 0) and only a few examples of an SAE (class 1). The large number of class 0 examples typically means that the accuracy of the model in predicting class 0 correctly, e.g., high specificity, is less important.

$$Specificity = \frac{T N}{T N + F P}$$

Key to the calculation of precision and recall is that the calculations do not make use of the true negatives. The calculations are concerned only with the correct prediction of the minority class, class 1.

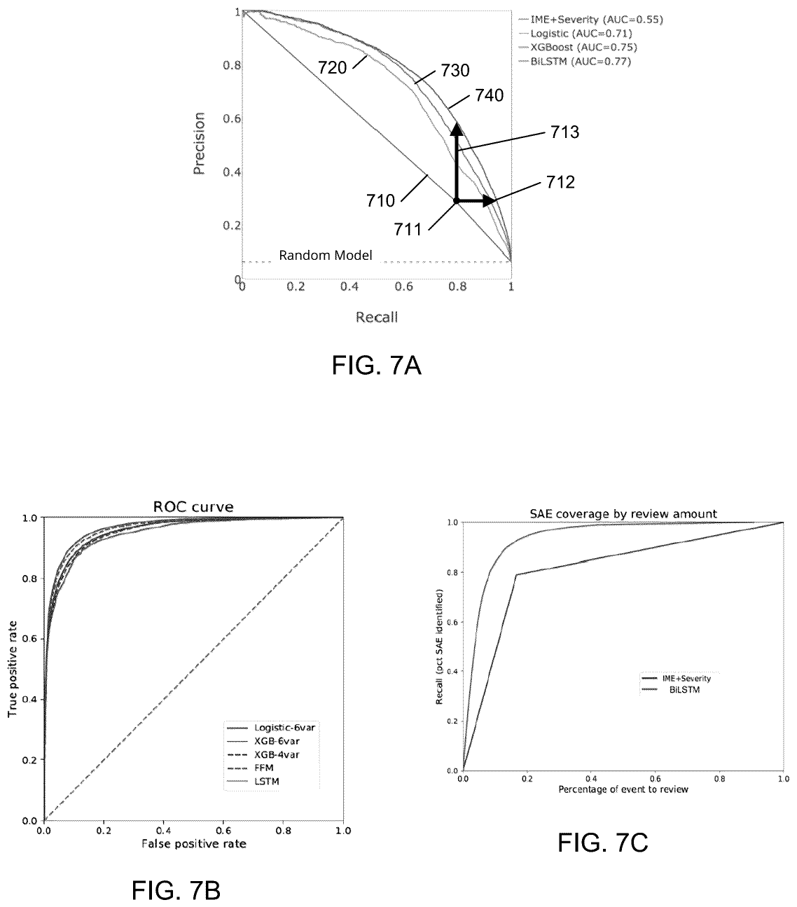

FIG. 7A shows precision-recall curves for a Random Model, the benchmark model, and various tested models. The Random Model has a precision of approximately 0.06 (shown by the dashed line). This is equal to the SAE rate in the training set data (=P/P+N). (If everything were predicted to be serious, Precision would equal this value, so it places a lower bound on desired performance of a model.) The benchmark model (“IME+severity”) determines that an adverse event is serious if the AE is on the IME list or the severity of the AE is Grade 3 (severe) or higher on the CTCAE scale. One point for the benchmark model is calculated to have a precision of 0.29 and a recall of 0.79, as shown by point 711, and PR curve 710 may be plotted. The area under PR curve 710 (AUC) is 0.55.

The other curves represent three tested models: a logistic regression model is indicated by 720, an XG Boost model is indicated by 730, and a Bi-LSTM model is indicated by 740. The AUC for the logistic regression model is 0.71, for the XG Boost model is 0.75, and for the Bi-LSTM model is 0.77. Thus, all three probabilistic models perform much better than the IME+Severity baseline (AUC=0.55), and the Bi-LSTM model has the best performance. Arrow 712 indicates that for the same precision (0.29) in the benchmark model, recall increases from 0.79 to 0.95 using the Bi-LSTM model. Similarly, arrow 713 indicates that for the same recall (0.79) in the benchmark model, precision increases from 0.29 to 0.60 using the Bi-LSTM model.

Performance of each model may also be made by examining the area under the ROC curve. The ROC curve plots the true positive rate vs. the false positive rate. Below is a table comparing area under the ROC curve for various numbers of variables (features) used with the logistic regression (“log”) and XGBoost (“xgb”) models. These results came from testing the validation set:

| Features | AUC (log) | AUC (xgb) |
|---|---|---|
| Severity, PT | 0.941 | 0.947 |
| Severity, PT, age | 0.941 | 0.948 |
| Severity, PT, indication | 0.946 | 0.953 |
| Severity, PT, indication, average | 0.947 | 0.956 |
| previous seriousness | | |
| Severity, PT, indication, outcome, | 0.949 | 0.958 |
| average previous seriousness | | |
| Severity, PT, indication, outcome, | 0.950 | 0.957 |
| average previous seriousness, age | | |
| Severity, PT, indication, outcome, | 0.944 | 0.958 |
| average previous seriousness, sponsor name | | |

With six variables, the XGBoost model performed better than the logistic regression model by 0.957/0.958 to 0.950. Note that using the sponsor name rather than age as a variable made the logistic regression model perform worse, but the same variable change made the XGBoost model perform better. While adding variables generally made the models perform the same or better, sometimes they made it worse (e.g., adding age as a third variable to the XGBoost model increased AUC by 0.001, whereas adding age as a sixth variable to the same model decreased AUC by 0.001).

The actual ROC curves for a number of models are shown in FIG. 7B. That graph compares the ROC curves for logistic regression-6 variable, XGBoost-6 variable, XGBoost-4 variable, feed-forward, and Bi-LSTM models. The performance of all of these models is good, with the Bi-LSTM model performing the best. The following table shows the AUC values for the five models in FIG. 7B plus a different XGBoost 6-variable model and the benchmark model on both the validation set and the test set, as well as accuracy for each model (where records with a prediction >0.5 are labeled as serious):

| | Validation | Hold-out Test Set | |
|---|---|---|---|
| Model | AUC | AUC | Accuracy |
| IME list | 0.729 | 0.734 | 88.6% |
| Logistic regression - 6 variables | 0.950 | 0.948 | 96.3% |
| (with outcome) | | | |
| XGB - 6 variables (same | 0.957 | 0.957 | 96.4% |
| variables as in logistic) | | | |
| XGB - 4 variables | 0.956 | 0.954 | 96.3% |
| XGB - 6 variables (with outcome | 0.958 | 0.959 | 96.5% |
| and sponsor) | | | |
| Feed-forward model | 0.966 | 0.964 | 96.6% |
| Bi-LSTM model with attention | 0.968 | 0.968 | 96.6% |
| to history | | | |

This table shows the substantial performance increase of the models over the benchmark model.

Another measure of performance is SAE coverage by review amount. As discussed earlier, medical reviewers can use the model estimated SAE likelihood to prioritize their review. The following simulation analysis was performed to show the advantage of doing so. The events in the test set are ordered either by the IME+Severity grade benchmark or by the SAE likelihood estimated by the Bi-LSTM model. Then the percentage of SAEs are computed among all SAEs in the dataset that are contained in the riskiest 10%, 20%, . . . adverse events ranked by the different models. In other words, this metric shows that to identify x % of SAEs in the dataset (x % is the same as recall), y % of all AEs need to be reviewed. FIG. 7C shows SAE coverage by review amount for the benchmark model and the bi-directional LSTM model, according to embodiments of the present invention. As shown in FIG. 7C, fewer adverse events need to be reviewed at all levels of recall when ranked by the Bi-LSTM model result compared to the IME+Severity grade benchmark. And to achieve 99% recall, only the riskiest 41% of AEs need to be reviewed under the Bi-LSTM model, rather than 96% events to be reviewed under the benchmark model. The following table shows some of these results at specific coverage levels:

| SAE coverage | Estimated review amount | |
|---|---|---|
| (Recall) | IME + severity | BiLSTM model |
| 99% | 96% | 41% |
| 95% | 80% | 21% |
| 90% | 60% | 14% |

This efficiency translates into real cost savings. For example, in a typical, multi-year, phase 3 clinical trial containing 5000 subjects, a senior medical reviewer may review 36,000 adverse events each year. The time to review each AE is one to two minutes. If the estimated percent reduction in reviews is 30-50%, the reviewer may save between 150 and 500 hours per year per trial. Based on a reviewer annual salary of $200,000, the annual reviewer time savings per trial can be from $15,000-$48,000.

Referring again to FIG. 4B, once the SAE model is assessed in operation 442, it may be refined in operation 452. One way to refine the model is try a different hyper-parameter set. Once the model has been refined, the final SAE model is generated in operation 454 using the training and validation sets. Then, operation 456 assesses the performance of the model on the held-out test set to obtain an estimate of performance of the final model on unseen data, i.e., a “real world” performance test set. This assessment differs from the one in operation 442 that is performed on the validation set to determine optimal hyper-parameters. In one embodiment (not shown in FIG. 4B), this final model may be retrained on the training, validation, and test sets (i.e., all data available) leading to an ultimate final model.

Referring back to FIG. 4A, the production workflow operates in a fashion similar to that of the development workflow. Data from a clinical trial that is being analyzed are collected via EDC 411. Clinical data extractor 412 extracts the relevant clinical data from the EDC, i.e., eCRF data 413. These data are then input to clinical data standardizer 415 to standardize the data and form and field names across trials using the form and field classifier so that the data from the forms and fields can be pooled together for analysis, similar to what standardizer 405 does. This time, however, the standardizer generates (S)AE data 418 and demographic (DM) data 416 from the clinical trial. (S)AE data 418, which includes serious and non-serious adverse event data from the clinical trial, are input to data processor 425. This processor operates like data processor 420 to automatically map the verbatim narratives to terms in a terminology dictionary, yielding standardized (S)AEs 435. Standardized (S)AEs 435 are input to SAE model 450, along with trial features 419, which come from a table containing curated trial-level data 417, and DM data 416 from the trial. SAE model 450 then determines the standardized (S)AEs along with the probability of each adverse event being serious 460, and provides information on the factors that contributed to that determination. User interface 470 then displays the list of adverse events ranked by the probability of being serious.

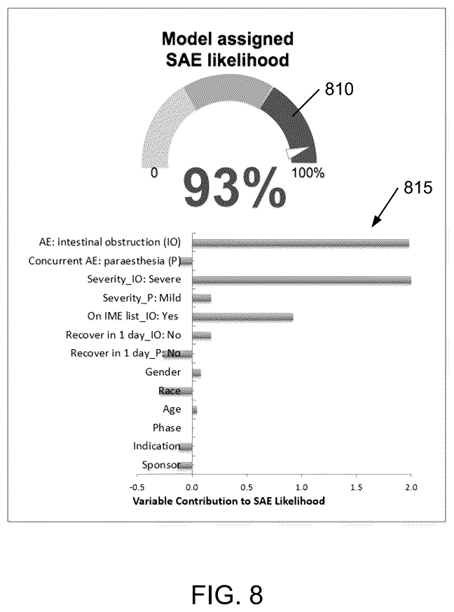

Several results of using the invention are shown in FIG. 8, which is an example of how the results are presented in a user interface. Item 810 shows the likelihood, assigned by the model, that the adverse event is considered serious, in this case, 93%. Item 815 shows possible features that factor into the determination of likelihood of seriousness. These features include the AE itself and whether there is a concurrent AE; the severity of the AE and concurrent AE; whether the AE and concurrent AE are on the IME list; whether the subject recovered in one day from the AE or the concurrent AE; several demographic categories; and the phase, indication, and sponsor of the clinical trial. Item 815 then shows the amount of contribution of each of the features. In this example, the AE itself (“intestinal obstruction” or “IO”) and the severity of the AE (“serious”) contribute the most to the seriousness determination—about 2.0 units (where the x-axis shows change of log-odds value of the predicted probability). In this case, “severity” is measured on the CTCAE scale described above. Whether the AE is on the IME list contributes the next most (˜1.0 unit). After those three features, no feature contributes more than 0.25 units, with several features (concurrent AE (“paraesthesia”) race, indication, sponsor, and whether recovered in 1 day for the concurrent AE) negatively contributing (tending to make the determination less serious) to the seriousness determination.

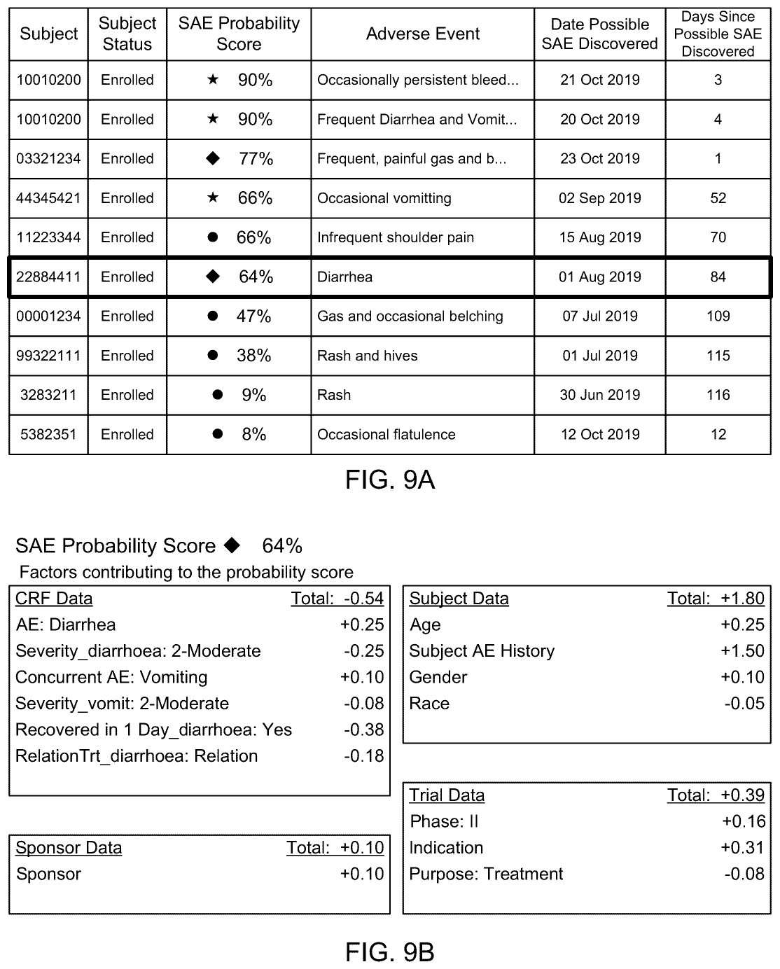

More results of the invention are shown in FIGS. 9A and 9B, which show how the invention may be used to prioritize AE review by diving deeper into the decision factors. FIG. 9A is a table listing the probabilities that adverse events may be considered serious. The invention allows the user to list the events in order of probable seriousness, so as to address the more serious adverse events first. In FIG. 9A, the SAE probability score ranges from 90% to 8%. The table includes a symbol (star, diamond, circle), to categorize the events into high/medium/low priority groups for review, where, for example, star=high, diamond=medium, and circle=low. Also included in the table are Subject No., Subject Status (whether the subject is currently enrolled or not, e.g., withdrawn), the description of the adverse event, the date the adverse event was discovered, and the number of days since the adverse event was discovered.

The invention also allows the user to review the factors contributing to the SAE probability score. FIG. 9B shows the factors underlying the “Diarrhea” adverse event in FIG. 9A. In the embodiment shown in FIG. 9B, four types of data may be used: CRF data (case report form data about an adverse condition for the subject of the clinical trial), demographic data for the subject, profile data for the clinical trial, and sponsor data.

CRF data may include the adverse event, its severity, whether there is a concurrent adverse event (and the severity of that AE), how long the AE lasted, and whether there is a relationship between the adverse event and the treatment that was under investigation in that particular trial. Demographic data for the subject may include age, subject adverse event history, gender, race, etc. Profile data for the clinical trial may include phase (I, II, or III), indication (e.g., diabetes, breast cancer, pneumonia, etc.), and purpose (e.g., treatment, prevention, diagnostic, supportive care, screening, health services research, and basic science). Sponsor data may include the name of the sponsor. For each of these factors, there may be associated an increase or a decrease in the SAE probability score (using change of log-odds units). In this embodiment, a subject that experienced moderately severe diarrhea, diarrhea provides an increase of 0.25 units, the severity is considered “moderate,” which provides a decrease of 0.25 units, concurrent vomiting provides an increase of 0.10 units, the “moderate” severity of the vomiting provides a decrease of 0.08 units, that the diarrhea cleared up in a day provides a decrease of 0.38 units, and the relationship between diarrhea and the treatment under test provides a decrease of 0.18 units.

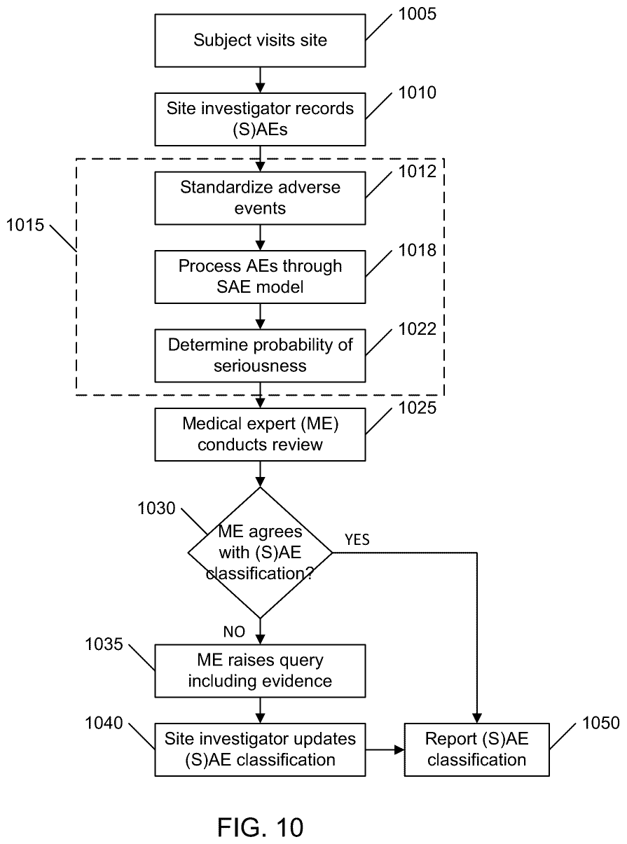

Reference is now made to FIG. 10, which is a flowchart similar to that of FIG. 1, showing how the SAE model of the present invention can be integrated into the AE review process. Operations 1005 and 1010 are the same as operations 105 and 110 in FIG. 1. Block 1015, which contains operations 1012, 1018, and 1022, represents modifications according to the present invention. In operation 1012, the system standardizes the adverse events as performed in operation 336 and by data processors 320, 420, and 425. In operation 1018, the SAE model processes the adverse events as described in connection with block 450. In operation 1022, the system determines the probability that an adverse event is serious as described in connection with block 460 and FIG. 8. With that information, in operation 1025, a medical expert reviews the seriousness determinations. Operation 1030 asks whether the medical expert agrees with the seriousness determination made in operation 1022. If so, then in operation 1050 the medical expert reports the adverse event determination. If the medical expert does not agree with the determination, then operations 1035 and 1040 are performed as in FIG. 1.

Besides the operations shown in FIGS. 1, 4B, and 10, other operations or series of operations are contemplated to automatically determine serious adverse events. Moreover, the actual order of the operations in the flowcharts in FIGS. 1, 4B, and 10 is not intended to be limiting, and the operations may be performed in any practical order.

The results illustrated above address some of the shortcomings stated above of the prior art method of determining seriousness of adverse events. For example, as discussed above, review of adverse events using this invention takes less time because, in the example above, only 41% of the adverse events would need to be reviewed to capture 99% of all of the SAEs, whereas 96% of the adverse events in the prior scheme would need to be reviewed to capture the same percentage. Second, this scheme reduces systemic and human error because it is more objective. Third, this scheme considers many more factors than the prior scheme could consider, e.g., whether there is a concurrent AE, the severity of the concurrent AE, and whether the subject recovered from the AE and/or the concurrent AE in one day, as well as demographic and trial information, such as gender, race, age, phase, indication, phase, and sponsor. Fourth, this scheme uses quantitative evidence, whereas in the prior scheme, such evidence was often lacking.

The invention also contributes to safety. In the case of the diabetes drug Avandia, in the prior scheme, three stroke events, which the FDA considers to be SAEs, were not reported as SAEs because the subjects were not hospitalized. See, “FDA Briefing Information, AVANDIA (Rosiglitazone Maleate) Tablets, for the Jul. 13-14, 2010 Joint Meeting of the Endocrinologic and Metabolic Drugs Advisory Committee and Drug Safety and Risk Management Advisory Committee” (accessible at wayback.archive-it.org/7993/20170112032224/http://www.fda.gov/downloads/AdvisoryCommittees/CommitteesMeetingMaterials/Drugs/EndocrinologicandMetabolicDrugsAdvisoryCommittee/UCM218493.pdf). In contrast, this inventive scheme does not rely on hospitalization to determine whether an adverse event is considered serious.

In sum, the invention includes machine learning models trained on a large amount of historical data, combining adverse event-, subject-, and trial-level information. It is able to distinguish SAEs from non-SAEs with high accuracy, evaluated by area under the ROC curve metric. The machine-learning models include logistic regression, extreme gradient boosting, and deep learning models. The advantages of the present invention over the prior methods include: (1) using a large amount of standardized AE data across sponsors reduces individual investigator and/or sponsor's inconsistencies in identifying SAEs; (2) using a machine-learning approach to model complicated relationships across factors associated with the adverse event, the subject, and the trial improves the accuracy of SAE identification; and (3) using the model-estimated SAE likelihood to prioritize events more likely to be serious for review reduces the review workload.

Aspects of the present invention may be embodied in the form of a system, a computer program product, or a method. Similarly, aspects of the present invention may be embodied as hardware, software or a combination of both. Aspects of the present invention may be embodied as a computer program product saved on one or more computer-readable media in the form of computer-readable program code embodied thereon.

The computer-readable medium may be a computer-readable storage medium. A computer-readable storage medium may be, for example, an electronic, optical, magnetic, electromagnetic, infrared, or semiconductor system, apparatus, or device, or any combination thereof.

Computer program code in embodiments of the present invention may be written in any suitable programming language. The program code may execute on a single computer, or on a plurality of computers. The computer may include a processing unit in communication with a computer-usable medium, where the computer-usable medium contains a set of instructions, and where the processing unit is designed to carry out the set of instructions.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

1. A method to train a serious adverse event model to detect serious adverse events from Electronic Data Capture (EDC) data for clinical trials, the method comprising:
extracting clinical data from EDC data of a plurality of clinical trials, the clinical data comprising form names and field names;
standardizing the clinical data across the plurality of clinical trials to generate subject-level data and adverse event data;
standardizing the adverse event data to produce standardized adverse events; and
training a serious adverse event model by inputting the standardized adverse events, trial-level features, and the subject-level data, wherein the trained serious adverse event model is adapted to determine a probability that each of the standardized adverse events is a serious adverse event.

2. The method of claim 1, wherein said training the serious adverse event model comprises inputting the standardized adverse events, trial-level features, and the subject-level data to a bidirectional Long Short-Term Memory (Bi-LSTM) neural network.

3. The method of claim 1, wherein said training the serious adverse event model comprises:
embedding an input vector comprising: a subvector derived from the standardized adverse event, a subvector derived from the trial-level features, and a subvector derived from the subject-level data.

4. The method of claim 3, wherein the input vector further comprises a weighted factor of historical adverse event scores for a corresponding subject in a corresponding clinical trial determined based at least in part on previous events experienced by said corresponding subject in said corresponding clinical trial.

5. The method of claim 4, wherein the weight of the weighted factor is determined based at least in part on a similarity metric between the historical adverse event and the standardized adverse event.

6. The method of claim 1, wherein, in said training the serious adverse event model, the standardized adverse events which occurred within a defined time period are used in sequential inputs to the Bi-LSTM neural network.

7. The method of claim 1, wherein said training the serious adverse event model comprises:
updating model weights in a Bi-LSTM neural network by optimizing the predicted likelihood against the observed seriousness label; and
generating a serious adverse event likelihood score based at least in part on the forward and backward hidden representations for each serious adverse event.

8. The method of claim 1, wherein said standardizing of the clinical data is performed using a trained classifying model.

9. The method of claim 1, wherein said standardizing of the adverse event data is performed using a trained classifying model comprising a terminology dictionary.

10. The method of claim 1, wherein the subject-level data comprise one or more of: age, gender, race, concurrent adverse events experienced by the corresponding subject in the corresponding trial within a defined time period, and previous events experienced by the corresponding subject in the corresponding trial.

11. The method of claim 1, wherein the trial-level features comprise one or more of: indication, phase, sponsor, therapeutic area, and primary purpose.

12. A method to detect serious adverse events from Electronic Data Capture (EDC) data for clinical trials using a trained serious adverse event model, the method comprising:
extracting clinical data from EDC data from a clinical trial, the clinical data comprising form names and field names;
standardizing the clinical data across a plurality of clinical trials to generate data and adverse event data of the clinical trial;
standardizing the adverse event data of the clinical trial to produce standardized adverse events of the clinical trial; and
processing, in a trained serious adverse event model derived from the plurality of clinical trials, the standardized adverse events of the clinical trial, trial-level features, and the subject-level data to determine a probability that each of the standardized adverse events of the clinical trial is a serious adverse event.

13. The method of claim 12, wherein said processing, in the trained serious adverse event model derived from the plurality of clinical trials, comprises inputting the standardized adverse events of the clinical trial, trial-level features, and the subject-level data of the clinical trial to a bidirectional Long Short-Term Memory (Bi-LSTM) neural network.

14. The method of claim 12, wherein said processing, in the trained serious adverse event model derived from the plurality of clinical trials, comprises:
embedding an input vector comprising: a subvector derived from a standardized adverse event, a subvector derived from the trial-level features, and a subvector derived from the subject-level data of the clinical trial.

15. The method of claim 14, wherein the input vector further comprises a weighted factor of historical adverse event scores for a corresponding subject determined based at least in part on previous events experienced by said corresponding subject in the clinical trial.

16. The method of claim 15, wherein the weight of the weighted factor is determined based at least in part on a similarity metric between the historical adverse event and the standardized adverse event of the clinical trial.

17. The method of claim 12, wherein, in said processing, in the trained serious adverse event model derived from the plurality of clinical trials, the standardized adverse events of the clinical trial which occurred within a defined time period are used in sequential inputs to the Bi-LSTM neural network.

18. The method of claim 12, wherein the serious adverse event model derived from the plurality of clinical trials is trained by a method comprising:
updating model weights in a Bi-LSTM neural network by optimizing the predicted likelihood against an observed seriousness label; and
generating a serious adverse event likelihood score based at least in part on the forward and backward hidden representations for each serious adverse event.

19. The method of claim 12, wherein said standardizing of the eCRF data is performed using a trained classifying model.

20. The method of claim 12, wherein said standardizing of the adverse event data of the clinical trial is performed using a trained classifying model comprising a terminology dictionary.

21. The method of claim 12, wherein the subject-level data comprise one or more of: age, gender, race, concurrent events experienced on a same day as an adverse event, and previous events experienced.

22. The method of claim 12, wherein the trial-level features comprise one or more of: indication, phase, sponsor, therapeutic area, and primary purpose.

23. The method of claim 12, further comprising displaying, via a user interface, a list of the standardized adverse events of the clinical trial ranked by determined probabilities of the standardized adverse events.

24. The method of claim 23, further comprising displaying, via the user interface, factors contributing to the determined probabilities.

25. A non-transitory computer-readable medium storing instructions that, when executed by one or more processors of a computer, cause said one or more processors to perform:
extracting clinical data from EDC data of a plurality of clinical trials, the clinical data comprising form names and field names;
standardizing the clinical data across the plurality of clinical trials to generate subject-level data and adverse event data;
standardizing the adverse event data to produce standardized adverse events; and
training a serious adverse event model by inputting the standardized adverse events, trial-level features, and the subject-level data, wherein the trained serious adverse event model is adapted to determine a probability that each of the standardized adverse events is a serious adverse event.

26. The non-transitory computer-readable medium of claim 25, wherein said training the serious adverse event model comprises:
embedding an input vector comprising: a subvector derived from the standardized adverse event, a subvector derived from the trial-level features, and a subvector derived from the subject-level data.

27. A non-transitory computer-readable medium storing instructions that, when executed by one or more processors of a computer, cause said one or more processors to perform:
extracting clinical data from EDC data from a clinical trial, the clinical data comprising form names and field names;
standardizing the clinical data across a plurality of clinical trials to generate data and adverse event data of the clinical trial;
standardizing the adverse event data of the clinical trial to produce standardized adverse events of the clinical trial; and
processing, in a trained serious adverse event model derived from the plurality of clinical trials, the standardized adverse events of the clinical trial, trial-level features, and the subject-level data to determine a probability that each of the standardized adverse events of the clinical trial is a serious adverse event.

28. The non-transitory computer-readable medium of claim 27, wherein said processing, in the trained serious adverse event model derived from the plurality of clinical trials comprises:
embedding an input vector comprising: a subvector derived from the standardized adverse event, a subvector derived from the trial-level features, and a subvector derived from the subject-level data.

29. A system to train a serious adverse event model to detect serious adverse events from Electronic Data Capture (EDC) data for clinical trials, the system comprising:
a computer having one or more processors in communication with a memory, the memory storing instructions executable by said one or more processors to perform:
extracting clinical data from EDC data of a plurality of clinical trials, the clinical data comprising form names and field names;
standardizing the clinical data across the plurality of clinical trials to generate subject-level data and adverse event data;
standardizing the adverse event data to produce standardized adverse events; and
training a serious adverse event model by inputting the standardized adverse events, trial-level features, and the subject-level data, wherein the trained serious adverse event model is adapted to determine a probability that each of the standardized adverse events is a serious adverse event.

30. The system of claim 29, wherein said training the serious adverse event model comprises:
embedding an input vector comprising: a subvector derived from the standardized adverse event, a subvector derived from the trial-level features, and a subvector derived from the subject-level data.

31. A system to detect serious adverse events from Electronic Data Capture (EDC) data for clinical trials using a trained serious adverse event model, the system comprising:
a computer having one or more processors in communication with a memory, the memory storing instructions executable by said one or more processors to perform:
extracting clinical data from EDC data from a clinical trial, the clinical data comprising form names and field names;
standardizing the clinical data across a plurality of clinical trials to generate data and adverse event data of the clinical trial;
standardizing the adverse event data of the clinical trial to produce standardized adverse events of the clinical trial; and
processing, in a trained serious adverse event model derived from the plurality of clinical trials, the standardized adverse events of the clinical trial, trial-level features, and the subject-level data to determine a probability that each of the standardized adverse events of the clinical trial is a serious adverse event.

32. The system of claim 31, wherein said processing, in the trained serious adverse event model derived from the plurality of clinical trials, comprises:
embedding an input vector comprising: a subvector derived from a standardized adverse event, a subvector derived from the trial-level features, and a subvector derived from the subject-level data of the clinical trial.
